(19) United States
(12) Patent Application Publication
(10) Pub. No.: US 2003/0104392 A1
(43) Pub. Date: Jun. 5, 2003

(54) Detection of growth performance of pigs

(76) Inventors: En-Chung Lin, Miaoli Hsien (TW); Ming-Yu Chen, Miaoli Hsien (TW); San-Yuan Huang, Miaoli Hsien (TW); Hui-Liang Tsou, Miaoli Hsien (TW); Wen-Chuan Lee, Miaoli Hsien (TW)

Correspondence Address: Edward M. Keating, COOK, ALEX, McFARRON,MANZO,CUMMINGS & MEHLER, LTD., Suite 2850, 200 West Adams Street, Chicago, IL, 60606, US

(21) Appl. No.: 09/995,026

(22) Filed: Nov. 27, 2001

(51) Int. Cl. C12Q 1/68

(52) U.S. Cl. 435/6

(57) ABSTRACT

The invention provides methods for selecting pigs with thin backfat, increased feed efficiency or reduced AGE through the identification of the nucleotide polymorphism of the 5′-flanking region of porcine HSP70.2 gene.

### BACKGROUND OF THE INVENTION

1. Field of the Invention

The present invention relates to the methods for detecting the growth performance such as backfat thickness, feed efficiency and AGE of pig.

2. Description of the Prior Art

The heat stress affects a pig in the backfat thickness, feed efficiency, growth and reproduction. The heat stress is caused by the following factors: temperature, relative humidity and solar radiation. The methods of reducing negative effects of heat stress on the pig are to change nutrition components, housing design and management. However, such methods only have limited effects and resulted in the increase of production costs. Therefore, it is necessary to develop more effective methods to improve resistance to heat stress in pigs.

A number of studies show that the heat shock protein 70 gene (HSP70 gene) is responsible for the tolerance to heat stress in multi-cell organisms. The promoter of HSP70 gene can regulate the expression of the functional gene. Peelman et al. firstly found the complete nucleotide sequence of HSP70.2 gene (Peelman et al., 1992, Immunogenetics 35, 286-9). Further studies reveal that the HSP70.2 gene promoter has additive genetic effects on pork quality (Schwerin et al., 1999, Arch. Tierz. 42 (Special Issue):61-66.). For example, the polymorphisms in the porcine HSP70.2 gene promoter are associated with performance traits. Schwerin et al. found 13 mutation sites from four different breeds and discovered TATA box affect pork quality in ways such as color and conductivity.

Up to now, there have been no effective techniques to utilize the polymorphism of the HSP70.2 gene to improve the growth performance. There is still a need to develop this technique in order to improve the growth performance of pigs under heat stress.

### SUMMARY OF THE INVENTION

One object of the invention is to provide a method for selecting pigs with thin backfat thickness, which comprises the step of identifying a polymorphism characterized by nucleotide position 393 of the 5′-flanking region of porcine Hsp70.2 gene, wherein the presence of the genotype of T/T at said position 393 indicates the pig with thin backfat thickness.

Another object of the invention is to provide a method for selecting the pigs with increased feed efficiency, which comprises the step of identifying a polymorphism characterized by nucleotide positions 44, 250 and 393 of the 5′-flanking region of porcine HsP70.2 gene, wherein the presence of the genotype of CAT/CAT or CAC/AAC at positions 44, 250 and 393 indicates the pigs with increased feed efficiency.

One further object of the invention is to provide a method for selecting pigs with reduced AGE (adjusted age at 110 kg body weight), which comprises the step of identifying a polymorphism characterized by nucleotide positions 250 and 393 of the 5′-flanking region of porcine Hsp70.2 gene, wherein the presence of the genotype of -C/AC at positions 250 and 393 indicates the pigs with reduced AGE.

### DETAILED DESCRIPTION OF THE INVENTION

The present invention features the use of the nucleotide polymorphism of the 5′-flanking region of porcine Hsp70.2 gene as the indicator of growth performance such as backfat thickness, feed efficiency and AGE of a pig. The invention provides the polymorphic BsaWI site in the 5′-untranslated leader sequence of porcine HsP70.2 gene. Such polymorphic sites can be used as an indicator to select the pig that has better growth performance such as backfat thickness, feed efficiency and AGE. In particular, the nucleotides at positions 44, 232, 250, 345 and 393 of the 5′-flanking region of porcine HsP70.2 gene are also associated with the growth performance of a pig.

Definition

The term “polymorphism” as used herein, refers to a variation in nucleotide sequence (and encoded polypeptide sequence, if relevant) at a given position in the genome within a population.

The term “single nucleotide polymorphism” (SNP), as used herein, refers to the occurrence of nucleotide variability at a single nucleotide position in the genome, within a population. A SNP may occur within a gene or within intergenic regions of the genome.

The term “5′-flanking region of porcine HSP70.2 gene”, as used herein, refers to upstream sequence of the porcine HsP70.2 gene which contained two consensus sequences of heat shock element along with two regions with homology to the CCAAT box, two binding sites for transcription factor SP1, and a TATA box. The 5′-untranslated leader sequence of the porcine HsP70.2 gene has a GC content of 64% and is 91% homogenous with the sequence reported by Peelman et al (Peelman et al., 1992, Immunogenetics 35, 286-9.)

The term “backfat thickness (BF),” as used herein, refers to a value measured on both sides of the following position: behind the scapula, last rib, and lumbar vertebra. BF was corrected for weight as follows (Chyr, S. C., 1980, J. Chin. Soc. Anim. Sci., 9: 55-69):

BF=average backfat thickness (mm)×[1+0.0088184×(110−end-test body weight (kg))]

The term “feed efficiency (FE),” as used herein, refers to a value calculated as:

FE=(total feed consumption during test)/(end-test body weight−start-test body weight)

The term “AGE,” as used herein, refers to an adjusted age at 110 kg body weight. AGE=end-test age−[(end-test body weight−110)/ADG]. ADG: average daily gain (kg/day), which is calculated as:

ADG(end-test body weight−start-test body weight)/(end-test age−start-test age)

Method for Detection of Backfat

The invention provides a method for selecting pigs with thin backfat thickness, which comprises the step of identifying a polymorphism characterized by nucleotide position 393 of the 5′-flanking region of porcine HsP70.2 gene, wherein the presence of the genotype of T/T at said position 393 indicates the pig with thin backfat thickness.

According to the invention, it is surprisingly found that the backfat thickness of pigs is associated with the nucleotide polymorphism at position 393 of the 5′-flanking region of porcine HsP70.2 gene. The identification of the genotype at position 393 of the 5′-flanking region of porcine HsP70.2 gene can be used in selecting the pigs with thin backfat thickness. The pigs with the genotype of T/T at position 393 of the 5′-flanking region of porcine HSP70.2 gene exhibit thin backfat.

Preferably, the method according to the invention comprises the step of identifying a polymorphism characterized by nucleotide positions 250 and 393 of the 5′-flanking region of porcine HSP70.2 gene, wherein the presence of the genotype AT/AT at positions 250 and 393 indicates the pig with thin backfat thickness. More preferably, said method comprises the step of identifying a polymorphism characterized by nucleotide positions 44, 250 and 393 of the 5′-flanking region of porcine HsP70.2 gene, wherein the presence of the genotype CAT/CAT at position 44, 250 and 393 indicates the pig with thin backfat thickness. Most preferably, the pigs with the genotype of CCATT/CCATT at positions 44, 232, 250, 345 and 393 exhibit thin backfat.

According to the invention, said pigs used in the invention can be selected from the group consisting of Duroc, Landrace and Yorkshire.

Method for Detection of Feed Efficiency

The invention also provides a method for selecting the pigs with increased feed efficiency, which comprises the step of identifying the polymorphism characterized by nucleotide positions 44, 250 and 393 of the 5′-flanking region of porcine HsP70.2 gene, wherein the presence of the genotype of CAT/CAT or CAC/AAC at positions 44, 250 and 393 indicates the pigs with increased feed efficiency.

According to the invention, it is surprisingly found that the feed efficiency of pigs is associated with the nucleotide polymorphism at positions 44, 250 and 393 of the 5′-flanking region of porcine HSP70.2 gene. The identification of the genotype at positions 44, 250 and 393 of the 5′-flanking region of porcine Hsp70.2 gene can be used in selecting the pigs with increased feed efficiency. The pigs with the genotype of CAT/CAT or CAC/AAC at positions 44, 250 and 393 exhibit increased feed efficiency. Most preferably, said method further comprises the step of identifying a polymorphism characterized by nucleotide positions 44, 250, 232 and 393 of the 5′-flanking region of porcine HSP70.2 gene, wherein the presence of the genotypes of CCATT/CCATT or CCATC/AAACC at positions 44, 232, 250, 345 and 393 indicates the pigs with increased feed efficiency.

According to the invention, said pigs used in the invention can be selected from the group consisting of Duroc, Landrace and Yorkshire.

Method for Detection of AGE

One further object of the invention is to provide a method for selecting pigs with reduced AGE, which comprises the step of identifying a polymorphism characterized by nucleotide positions 250 and 393 of the 5′-flanking region of porcine HsP70.2 gene, wherein the presence of the genotype of -C/AC at positions 250 and 393 indicates the pigs with reduced AGE.

According to the invention, it is surprisingly found that the days needed to reach 110 Kg for pigs is associated with the nucleotide polymorphism at positions 250 and 393 of the 5′-flanking region of porcine HSP70.2 gene. The identification of the genotype at positions 250 and 393 of the 5′-flanking region of porcine Hsp70.2 gene can be used in selecting the pigs with reduced AGE. The pigs with the genotype of -C/AC at positions 250 and 393 exhibit reduced AGE.

Preferably, the method according to the invention comprises the step of identifying a polymorphism characterized by nucleotide positions 44, 250 and 393 of the 5′-flanking region of porcine HsP70.2 gene, wherein the presence of the genotypes of C-C/CAC at positions 44, 250 and 393 indicates the pigs with reduced AGE. Most preferably, said method comprises the step of identifying a polymorphism characterized by nucleotide positions 44, 232, 250 and 393 of the 5′-flanking region of porcine HSP70.2 gene, wherein the presence of the genotypes of CC-TC/CCATC at positions 44, 232, 250, 345 and 393 indicates the pigs with reduced AGE.

According to the invention, said pigs used in the invention can be selected from the group consisting of Duroc, Landrace and Yorkshire.

Given the above, the genotypes of the nucleotide polymorphism of the 5′-flanking region of porcine HSP70.2 gene can be used in the detection of the growth performance of a pig.

The following Examples are offered by way of illustration and not by way of limitation.

### EXAMPLES

Animals

The 119 purebred Duroc pigs used in this study were obtained from 11 purebred farms in Taiwan. Genetic relationships among the pigs were avoided as much as possible. The pigs were sent to the Northern Central Testing Station at the Pig Research Institute Taiwan in 5 batches using the segregated early weaning (SEW) entrance method (July, September, November, December 1999, and March 2000). The weight and entry age were limited to 4-9 kg (5.94±1.35 kg in average) and 14-20 days (17.2±2.1 days in average), respectively. The tested piglets were raised in the modular SEW nursery (Double L Group Inc.) for about 42 days, then were moved to the testing house.

Performance Test

The tested pigs started the performance tests at a body weight of 30.0±2.0 kg. The tests were ended when the pigs reached a body weight of 110.0±2.0 kg. Each tested pig was kept in a pen of 4 m² in size. Altogether, there are 6 testing houses at the central testing station. Each house has 66 pens, allowing the animals from the same batch to be kept in the same house. All the tested pigs were fed ad libitum with the same diet of crude protein 18.5%, metabolizable energy 3,140 kcal/kg, calcium 1.20%, phosphorus 0.80%, Lysine 1.06%, Methionine 0.41%, Cystine 0.30%, and Tryptophan 0.20%. The temperature at the testing house and the daily feed intake were recorded daily. At the end of the performance test, there were four traits documented: days of age at test started (SAGE), average daily gain adjusted to 110 kg of body weight (ADG), feed efficiency (the ratio of total weight gain to total feed intake, FE), backfat thickness adjusted to 110 kg of body weight (BF), days of age to 110 kg (AGE).

DNA Isolation and Sequencing

Genomic DNA was isolated from blood of the tested pigs using a DNA Isolation Kit for manunalian blood (Boehringer Mannheim, Ind., USA). The primers used in this study were designed according to the HSP70 gene sequence reported by Peelman et al. (1992). The reaction conditions for PCR followed the procedure set up by Chen et al. (2001). The PCR products were purified from gels using a Gel Extraction Miniprep Kit (Viogene, Sunnyvale, Calif., USA), and used in the following sequencing. The purified PCR products were sequenced in both directions, and the nucleotide sequences were recorded with an automated DNA sequencer (ABI 377, Perkin-Elmer, Forster, Calif., USA).

Determination of Genotype

According to Chen et al. (2001), there were five single nucleotide polymorphisms (SNPs), nt44 (A/C), nt232 (A/C), nt250 (A/), nt345 (T/C), and nt393 (T/C), on the 5′-flanking region of porcine HSP70.2 gene of three major breeds, Duroc, Landrace, and Yorkshire, in Taiwan. To maximize the amount of information about the SNPs in this association study, haplotypes of the five SNPs in both chromosomes for each individual were decided by the genotypes of its siblings and/or parents. Thus, the genotype of that 5′-flanking region of porcine HSP70.2 gene for each individual could be determined by the two haplotypes. For example, the five SNPs of a particular individual have CC, CC, A-(− denotes deletion), TT, TC on the nt44, nt232, nt250, nt345, and nt393, respectively. There are two possibilities of haplotype combination, CCATT/CC-TC and CCATC/CC-TT. After checking haplotypes of its siblings and/or parents, we may decide which haplotype combination is the correct one as its genotype of this 5′-flanking region of porcine HSP70.2 gene.

Statistical Analysis

The four performance traits (ADG, FE, BF and AGE) were analyzed by using a linear model with SAGE as a covariate and season during the test, genotype, and interaction of both as fixed effects. The statistical analyses were conducted by using SAS GLM procedure (SAS Institute, 1989).

Results

Among the 119 tested Duroc pigs, there were 238 chromosomes with only 5 different haplotypes. The distribution of the five haplotypes is shown in Table 1. The percentage of haplotypes CCATT, CC TC, and CCATC add up to about 90%. The haplotype CCATC is the most of the frequent among the 119 animals. In contrast, the haplotype CC TT is the least frequent at 1.3%. From the results of the human genome project, Brookes (1999) defined the SNP as “Single base pair positions in genomic DNA at which different sequence alternatives (alleles) exist in normal individuals in some population(s), wherein the least frequency allele has an abundance of 1% or greater.” The 119 tested pigs were collected from 11 purebred farms around Taiwan. Thus, the distribution found in the tested pigs might represent the possible distribution of haplotypes in the purebred Duroc population in Taiwan. The five haplotypes show that if is “A” found at the nt44 position, there are a “A” and a “C” at the nt232 and nt345 positions respectively. If a “C” is found at the nt44 position; however, there are a “C” and a “T” at the nt232 and nt345 positions. Thus, the only two positions changed were nt250 and nt393.

TABLE 1

| Haplotype | CCATT | CC TC | CCATC | AAACC | CC TT | Total |
|---|---|---|---|---|---|---|
| Frequency | 29.0% | 18.1% | 42.9% | 8.8% | 1.3% | 100.0% |
| (Chromo- | (69) | (43) | (102) | (21) | (3) | (238) |
| some) | | | | | | |

Values in the parentheses are the number of chromosomes with the haplotypes indicated above. denotes a deletion of nucleotide in the position of nt250.

The various haplotype combinations resulted in 11 genotypes found in this population (Table 2). Among the 11 genotypes, there were 6 major genotypes, which accounted for up to 84.9% of the animals: these were CCATT/CCATT, CCATT/CC TC, CCATT/CCATC, CC TC/CCATC, CCATC/CCATC, and CCATC/AAACC. As the number of animals surveyed increases, there will be more possible genotypes. There were 91 animals tested with 6 genotypes (CCATT/CCATT, CCATT/CCATC, CCATT/AAACC, CC TC/CCATC, CCATC/CCATC, and CCATC/AAACC). The performance traits of those 91 animals were actually involved and compared in the statistical analysis.

TABLE 2

| Geno- | CCATT | CCATT | CCATT | CCATT | CCATT | CC TC |
|---|---|---|---|---|---|---|
| type | CCATT | CC TC | CCATC | AAACC | CC TT | CC TC |
| Fre- | 8.4% | 6.7% | 27.7% | 4.2% | 2.5% | 5.0% |
| quency | (10) | (8) | (33) | (5) | (3) | (6) |
| (head) | | | | | | |
| Geno- | CC TC | CC TC | CCATC | CCATC | AAACC | |
| type | CCATC | AAACC | CCATC | AAACC | AAACC | Total |
| Fre- | 17.6% | 1.7% | 16.0% | 8.4% | 1.7% | 100.0% |
| quency | (21) | (2) | (19) | (10) | (2) | (119) |
| (head) | | | | | | |

From Table 3, backfat thickness was the trait influenced most by the genotypes of the 5′-flanking region of porcine HSP70.2 gene (p<0.01). The genotypes of CCATC/CCATC and CCATC/AAACC significantly increased backfat tickness of pigs more than the other 4 genotypes. The trait of days of age to 110 kg (AGE) was also affected by genotype (p<0.05) The genotype CC TC/CCATC reduced the time to reach 110 kg of body weight about 8 days more than the genotype of CCATC/AAACC. The genotype of CCATT/CCATT had better feed efficiency (FE) than CCATC/CCATC (p<0.1).

TABLE 3

| Genotype** | FE | BF | AGE |
|---|---|---|---|
| CCATT/CCATT | 2.13 ± 0.06^{b} | 1.35 ± 0.04^{y} | 155.4 ± 2.2^{de} |
| (10) | | | |
| CCATT/CCAT | 2.24 ± 0.03^{ab} | 1.41 ± 0.02^{y} | 153.9 ± 1.3^{de} |
| C (32) | | | |
| CCATT/AAACC | 2.28 ± 0.08^{ab} | 1.40 ± 0.06^{xy} | 152.1 ± 3.2^{de} |
| (5) | | | |
| CC TC/CCATC | 2.18 ± 0.05^{ab} | 1.41 ± 0.03^{y} | 150.8 ± 1.8^{e} |
| (16) | | | |
| CCATC/CCATC | 2.25 ± 0.05^{a} | 1.52 ± 0.03^{x} | 154.3 ± 1.7^{de} |
| (18) | | | |
| CCATC/AAACC | 2.16 ± 0.06^{ab} | 1.53 ± 0.04^{x} | 158.7 ± 2.2^{d} |
| (10) | | | |

Values with superscription of a, b mean significant difference with p < 0.1; d, e, f with p < 0.05; x, y with p < 0.01.
**Values in the parentheses are the number of observations for those genotypes.

The effects of genotypes of the 5′-flanking region of 15 porcineHSP70.2 gene on BF, AGE, and FE are further determined in cold season (Table 4). For BF, FE and AGE, the differences among genotypes observed in Table 3 are actually found within cold season.

The animals with genotype of CCATT/CCATT had least thickness of BF in the cold weather. Those with CCATC/CCATC and CCATC/AAACC got the largest backfat thickness than the others. Those animals with CCATT/CCATC and CC TC/CCATC had intermediate backfat thickness.

Similar difference among genotypes within cold season was observed on the trait of AGE (p<0.05). The animals with CC TC/CCATCT had the reduced AGE. The animals with CCATC/AAACC have to spend 8.7-10.4 more days to reach 110 kg of body weight than those with genotypes of CCATT/CCATT, CCATT/CCATC, and CC TC/CCATC. But, the genotype of CCATT/CCATT spent 8.5 more days to reach 110 kg of body weight than those with CC TC/CCATC.

For the trait of FE, the animals with CCATC/AAACC had best figure than the others. Those animals with CCATC/CCATC and CCATT/CCATC had the worst FE among the genotypes, and those with CCATT/CCATT and CCATC/AAACC had the best FE among the genotypes.

TABLE 4

| Genotype | FE | BF | AGE |
|---|---|---|---|
| CCATT/CCATT | 2.07 ± 0.08^{ef} | 1.30 ± 0.06^{z} | 151.0 ± 3.1^{e} |
| (5, 5) | | | |
| CCATT/CCATC | 2.25 ± 0.04^{d} | 1.43 ± 0.03^{y} | 151.9 ± 1.5^{e} |
| (22, 10) | | | |
| CCATT/AAACC | 2.28 ± 0.10^{de} | 1.41 ± 0.07^{yz} | 152.8 ± 4.0^{de} |
| (3, 2) | | | |
| CC TC/CCATC | 2.20 ± 0.06^{de} | 1.47 ± 0.04^{y} | 150.2 ± 2.3^{e} |
| (9, 7) | | | |
| CCATC/CCATC | 2.34 ± 0.07^{d} | 1.61 ± 0.05^{x} | 155.9 ± 2.8^{de} |
| (6, 12) | | | |
| CCATC/AAACC | 2.01 ± 0.08^{f} | 1.70 ± 0.05^{x} | 160.6 ± 3.1^{d} |
| (5, 5) | | | |

*Values across the same column with superscription of d, e, f mean significant difference with p < 0.05; x, y, z with p < 0.01 Values between two seasons within a particular genotype for a trait with letters underneath of d, e mean significant difference with p < 0.05; x, y with p < 0.01.

What is claimed is:

1. A method for selecting pigs with thin backfat thickness, which comprises the step of identifying a polymorphism characterized by nucleotide position 393 of the 5′-flanking region of porcine HSP70.2 gene, wherein the presence of the genotype of T/T at said position 393 indicates the pig with thin backfat thickness.

2. A method of claim 1, wherein said pigs are selected from the group consisting of Duroc, Landrace and Yorkshire.

3. A method of claim 1, which further comprise the step of identifying a polymorphism characterized by nucleotide position 250 of the 5′-flanking region of porcine HSP70.2 gene, wherein the presence of the genotype AT/AT at positions 250 and 393 indicates the pig with thin backfat thickness.

4. A method of claim 3, which further comprises the step of identifying a polymorphism characterized by nucleotide position 44 of the 5′-flanking region of porcine HSP70.2 gene, wherein the presence of the genotype CAT/CAT at position 44, 250 and 393 indicates the pig with thin backfat thickness.

5. A method of claim 4, which further comprises the step of identifying a polymorphism characterized by nucleotide position 232 of the 5′-flanking region of porcine HSP70.2 gene, wherein the presence of the genotype CCATT/CCATT at position 44, 232, 250, 345 and 393 indicates the pig with thin backfat thickness.

6. A method for selecting the pigs with increased feed efficiency, which comprises the step of identifying a polymorphism characterized by nucleotide positions 44, 250 and 393 of the 5′-flanking region of porcine HSP70.2 gene, wherein the presence of the genotype of CAT/CAT or CAC/AAC at positions 44, 250 and 393 indicates the pigs with increased feed efficiency.

7. A method of claim 6, which further comprises the step of identifying a polymorphism characterized by nucleotide position 232 of the 5′-flanking region of porcine HSP70.2 gene, wherein the presence of the genotypes of CCATT/CCATT or CCATC/AAACC at positions 44, 232, 250, 345 and 393 indicates the pigs with increased feed efficiency.

8. A method of claim 6, wherein said pigs are selected from the group consisting of Duroc, Landrace and Yorkshire.

9. A method for selecting pigs with reduced AGE, which comprises the step of identifying a polymorphism characterized by nucleotide positions 250 and 393 of the 5′-flanking region of porcine HSP70.2 gene, wherein the presence of the genotype of -C/AC at positions 250 and 393 indicates the pigs with reduced AGE.

10. A method of claim 9, which further comprises the step of identifying a polymorphism characterized by nucleotide position 44 of the 5′-flanking region of porcine HSP70.2 gene, wherein the presence of the genotypes of C-C/CAC at positions 44, 250 and 393 indicates the pigs with reduced AGE.

11. A method of claim 9, which further comprises the step of identifying a polymorphism characterized by nucleotide position 232 of the 5′-flanking region of porcine HSP70.2 gene, wherein the presence of the genotypes of CC-TC/CCATC at positions 44, 232, 250, 345 and 393 indicates the pigs with reduced AGE.

12. A method of claim 9, wherein said pigs are selected from the group consisting of Duroc, Landrace and Yorkshire.
